(19) United States
(12) Patent Application Publication
(10) Pub. No.: US 2016/0339230 A1
(43) Pub. Date: Nov. 24, 2016

(54) Immobilization Systems for Treatment Applications

(71) Applicant: Hermo Medical Solutions, S.L., Zaragoza (ES)

(72) Inventors: Javier Muñoz, Zaragoza (ES); Toni Climent, Alicante (ES); Luis Monzón, Zaragoza (ES); Lucas Pedrajas, Zaragoza (ES); Juan Monzón, Zaragoza (ES)

(21) Appl. No.: 15/161,269

(22) Filed: May 22, 2016

Related U.S. Application Data
(60) Provisional application No. 62/165,428, filed on May 22, 2015.

(51) Int. Cl. A61N 1/04; A61B 5/00; A61F 5/058

(52) U.S. Cl. CPC A61N 1/0468; A61F 5/05841; A61N 1/0452; A61B 5/0022

(57) ABSTRACT

Immobilization systems are provided for treatment of an injured area. The system includes a splint for placement around an injured area, a therapeutic device coupled to the splint to effectuate treatment to target tissues around the injured area and a wireless interface configured to communicate with the device to transmit treatment data to the device. Methods for treatment of an injured area are also provided.

### CROSS REFERENCE TO RELATED APPLICATIONS

This application claims priority to and the benefit of U.S. Provisional Application Ser. No. 62/165,428, filed May 22, 2015, the disclosure of which is hereby incorporated herein by reference in its entirety.

### FIELD

The present disclosure relates to immobilization systems, and more particularly customized immobilization systems for treatment of an injured area.

### BACKGROUND

When bones are fractured, cracked, or ligaments are lengthened or ruptured, an orthopedic cast or splint is often applied to the injured area to immobilize the injured joints and muscles partially or entirely.

One issue with using splints or casts is that they can often not be easily applied to and removed from the injured area. For example, application of a plaster bandage can be complicated, and once the plaster bandage is placed over the injured area, it typically remains in place for about five weeks, which can promote the growth of mold or infectious bacteria. Further, the process of the removing cast by using a saw can generate dust, which can cause problems to the injured area.

Moreover, when the cast is applied around the injured area, it can be difficult to initiate early joint movement, and inaccurate or abnormal fixation cannot be checked through intermediate inspections due to the cast covering the injured area. Even after the splint or cast is removed, it can often be replaced with another type of splint, for the rehabilitation phase and can result in similar issues noted above. Since rehabilitation cannot be started until bone immobilization is completed, the application of a splint to the injured area can lead to muscular atrophy. Long recovery times can result in unnecessary costs to the injured person, since there are a number of the healthcare providers and other individuals (e.g., patients, employers, rehabilitation centers and health insurance companies) involved in the recovery process.

Thus, there is a need for an immobilization system that can overcome these and other issues.

### SUMMARY

In one aspect, a system for immobilization and treatment of an injured area is provided. The system comprises a splint for placement around an injured area, a therapeutic device coupled to the splint to effectuate treatment of target tissues, and a wireless interface configured to communicate with the therapeutic device to transmit treatment data to the device.

In some embodiments, the splint is sufficiently flexible to permit movement around the injured area. In some embodiments, the splint is perforated to promote air circulation, and to minimize microbial growth around the injured area. In some embodiments, the therapeutic device is designed to transmit progress data to the wireless interface. In some embodiments, the therapeutic device is an electrotherapeutic device. In some embodiments, the therapeutic device is a sensor to detect muscle mass index. In some embodiments, the wireless interface is configured to receive and store data received from the therapeutic device.

The system can, in some embodiments, transmit treatment data to the wireless interface from a remote location, prior to the wireless interface sending the treatment data to the therapeutic device. In some embodiments, the wireless interface can be designed to receive sensor data from the therapeutic device, which can be sent to a remote location for monitoring.

In another aspect, a method for orthopedic treatment is provided. The method comprises providing an immobilization system including a splint, designed from a three dimensional (3D) scan of a limb for placement around an injured area, and a therapeutic device coupled to the splint, which can be configured to effectuate treatment of target tissues around the injured area. The method further includes sending treatment data to the therapeutic device via a wireless interface to effectuate treatment, by action of the therapeutic device transmitting a stimulation signal to the target tissues.

In some embodiments, the stimulation signal is used to treat skeletal or muscle tissue. In some embodiments, the method allows the wireless interface to communicate with a health care provider's computer to inform the provider of treatment progress. In some embodiments, the method can also permit a provider to send additional treatment data to the wireless interface that can be transmitted to the therapeutic device for treatment.

In another aspect, a method for treatment of an injured area is provided. The method comprises identifying an injured region on a limb, placing markers on the limb around the injured region, and then scanning the injured region with markers to generate data. The data can be used to fabricate a splint that conforms to the features of the limb. The splint can be used to attach a therapeutic device and active the device wirelessly to treat the injured area.

### BRIEF DESCRIPTION

The presently disclosed embodiments will be further explained with reference to the attached drawings. The drawings shown are not necessarily to scale, with emphasis instead generally being placed upon illustrating the principles of the presently disclosed embodiments.

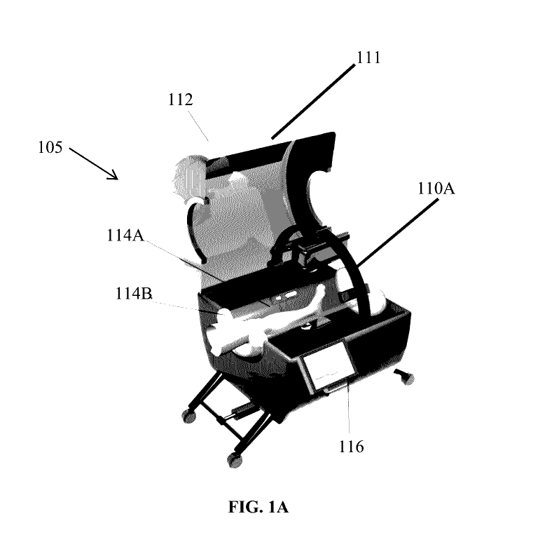

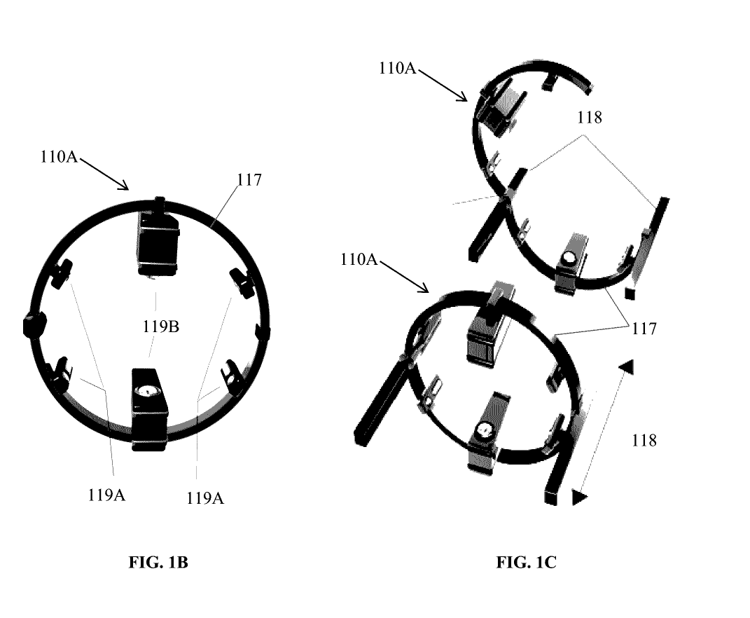

FIG. 1A, illustrates a fabrication system for generating a customized splint, according to embodiments of the present disclosure;

FIGS. 1B-1C illustrate a scanner for use with fabrication system, according to embodiments of the present disclosure;

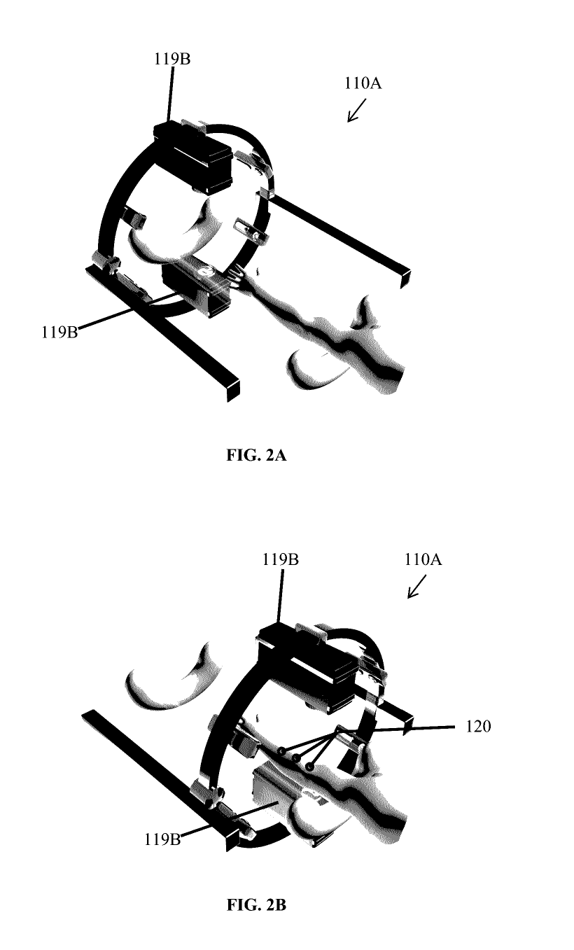

FIGS. 2A-2B illustrate an example of patient orientation during the scan procedure, according to embodiments of the present disclosure;

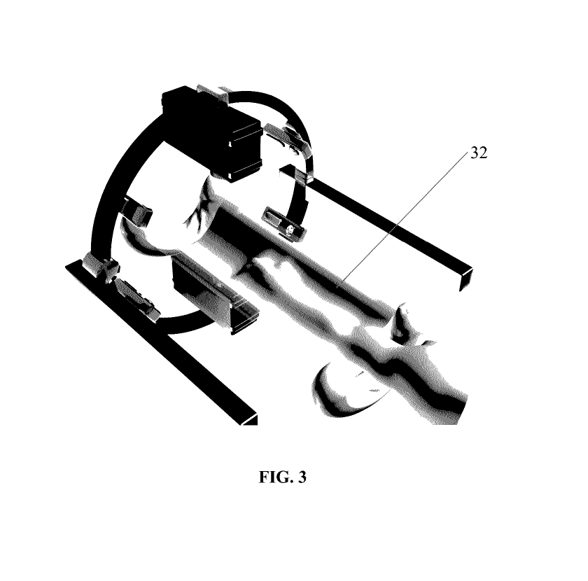

FIG. 3 illustrates a sheet of photo-polymeric placed over an injured body part, according to embodiments of the present disclosure;

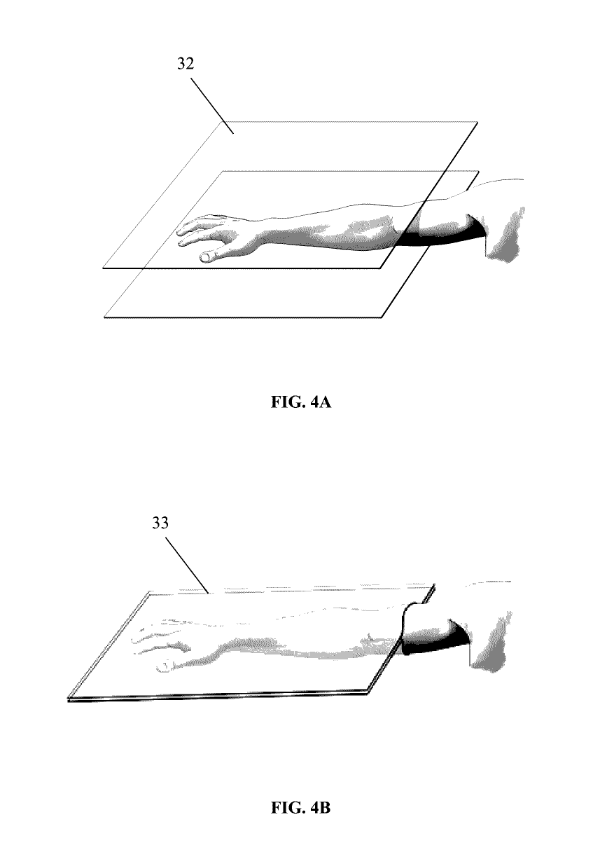

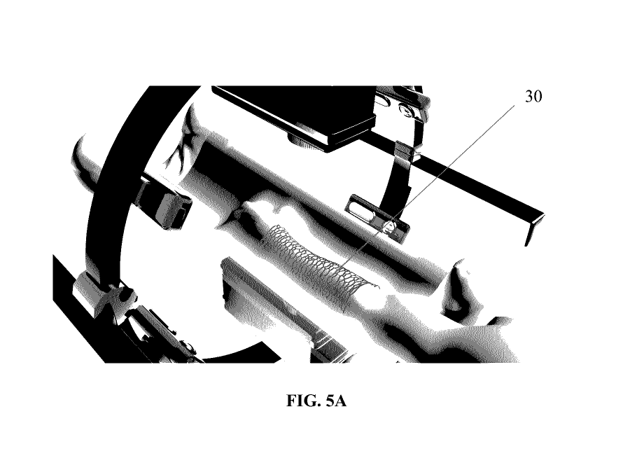

FIGS. 4A-4B illustrate the placement of a sheet of photo-polymeric material over the injured body part, according to embodiments of the present disclosure;

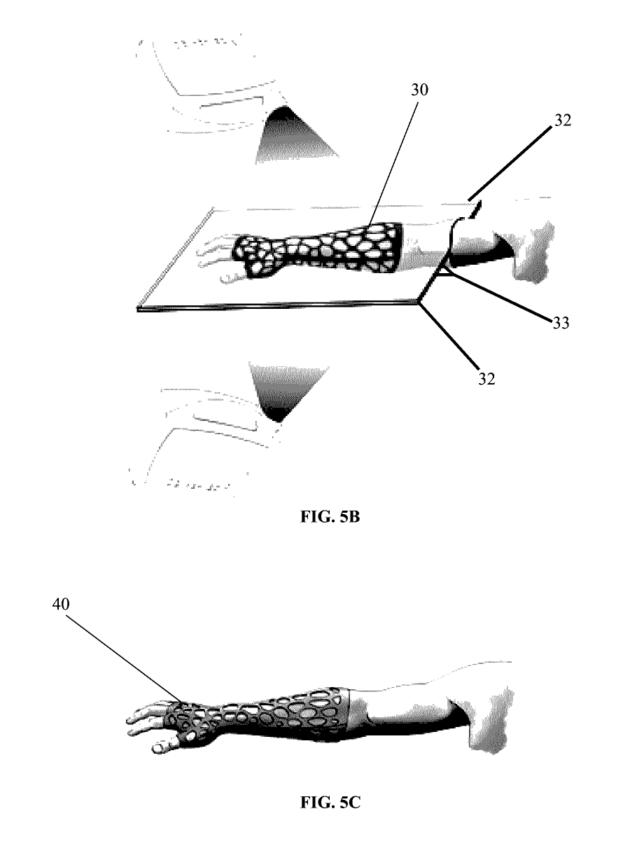

FIGS. 5A-5B illustrate the projected image of a to-be-fabricated splint onto the surface of the photo-polymeric material, according to embodiments of the present disclosure;

FIG. 5C illustrates a newly formed splint, according to embodiments of the present disclosure;

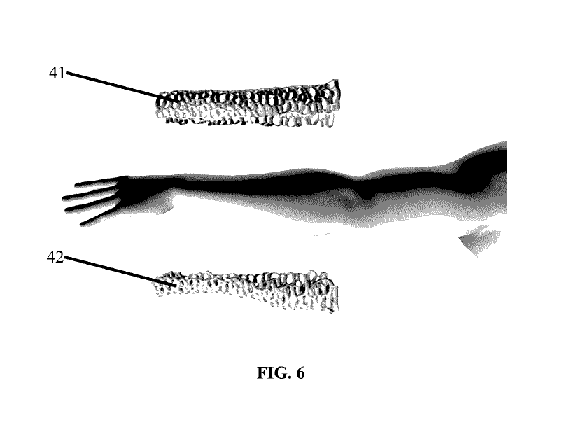

FIG. 6 illustrates two halves of the splint shown in FIG. 5C, according to embodiments of the present disclosure;

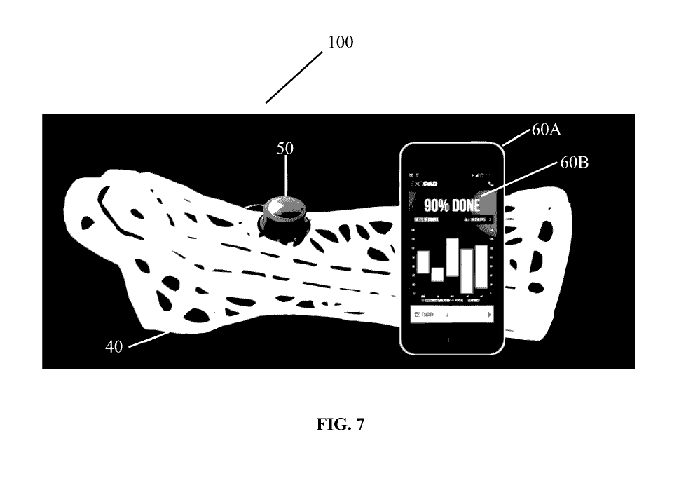

FIG. 7 illustrates components of an immobilization system according to embodiments of the present disclosure;

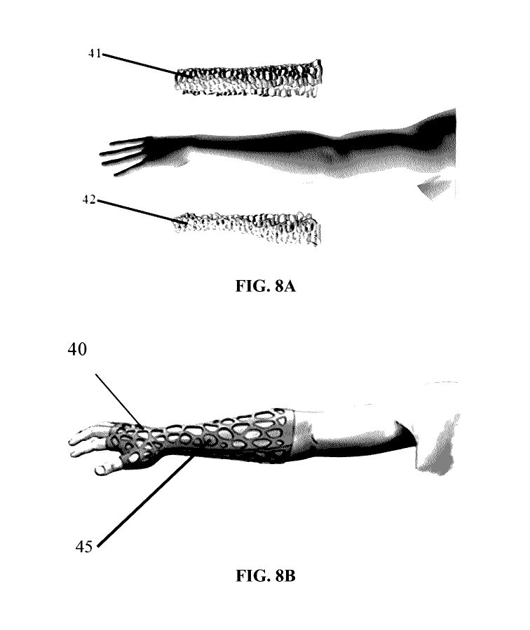

FIGS. 8A-8B illustrate a splint used in connection with the immobilization system in FIG. 7 in an unassembled state and assembled state attached to a body part, according to embodiments of the present disclosure;

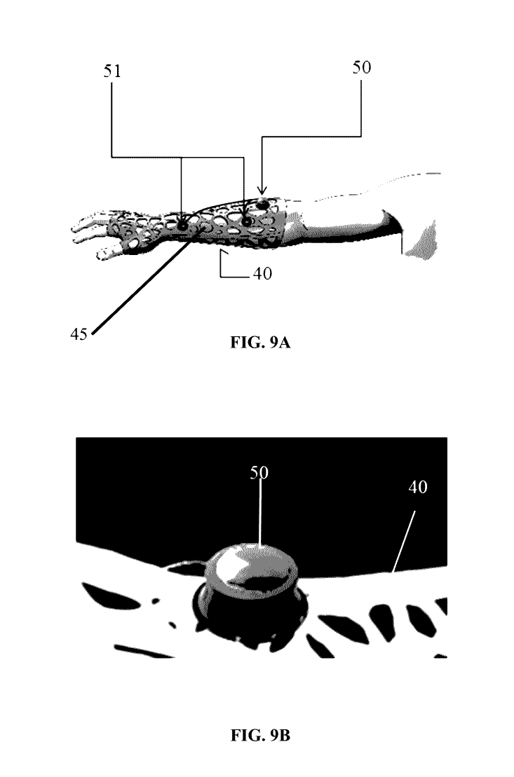

FIGS. 9A-9B illustrate a therapeutic device mounted onto the splint, according to embodiments of the present disclosure;

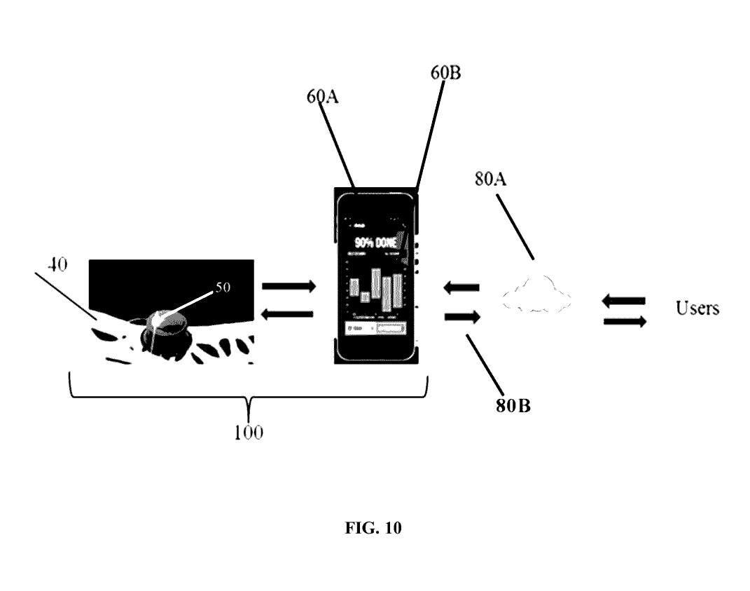

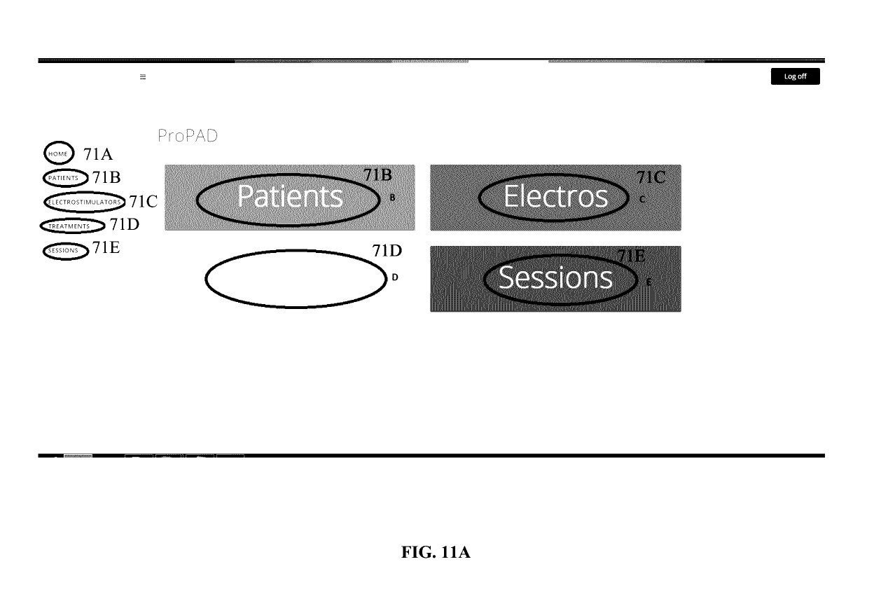

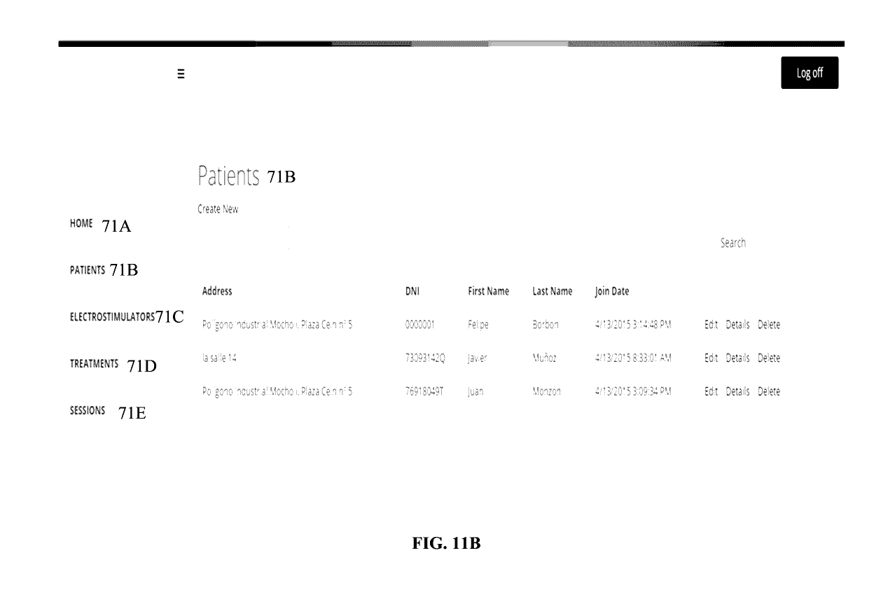

FIG. 10 illustrates a system designed to permit communication of information to and from the therapeutic device, according to embodiments of the present disclosure;

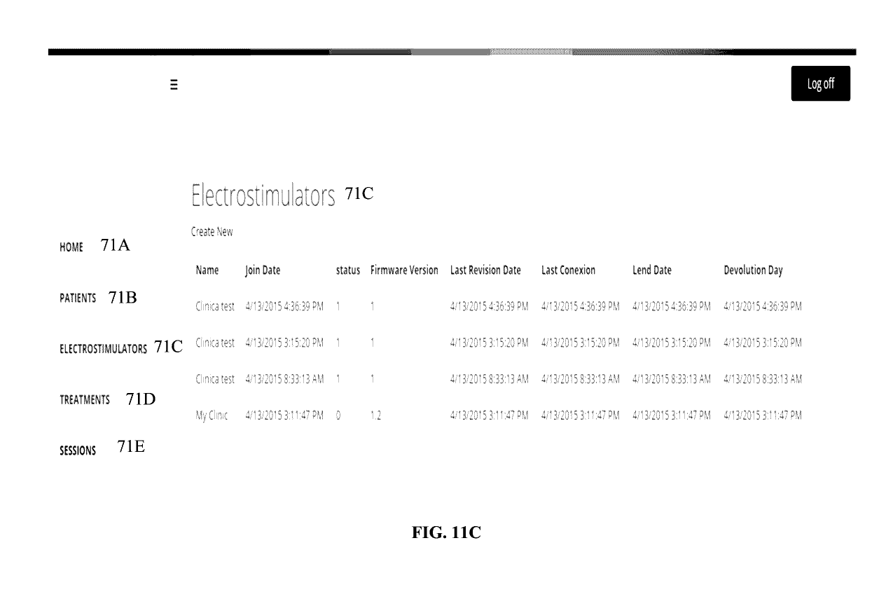

FIGS. 11A-11C illustrate aspects of a smart device application used in connection with the process of treatment, according to embodiments of the present disclosure; and

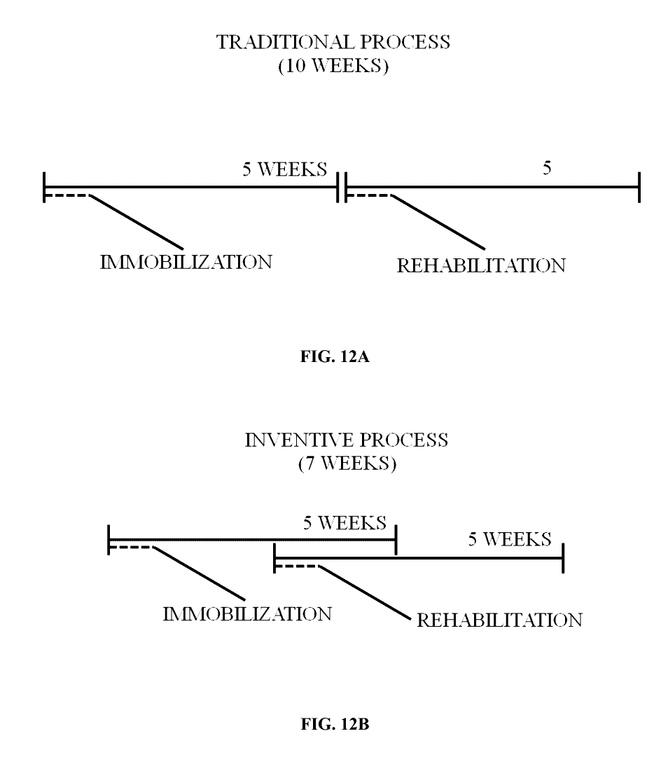

FIGS. 12A-12B illustrates a comparative timeline between a traditional healing process and the process of the present invention, according to embodiments of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure is directed to an immobilization system designed to improve processes for treating an injured area. The immobilization system can improve patient quality of life by improving the patient's healing processes through the use of a custom splint. The custom splint, in one embodiment, can be generated through the use of a fabrications system as described below.

### Splint Fabrication System

With reference to FIG. 1A, fabrication system 105, in one embodiment, includes a three dimensional (3D) scanner 110A for generating a three dimensional image of a region of interest (i.e. injured area) on a limb. Fabrication system 105 can also include a light filter 112 on lid 111, supports 114A and 114B, and a control screen 116 to control the operation of the system 105.

FIG. 1B and FIG. 1C illustrate, in one embodiment, scanner 110A for use with system 105. As illustrated, scanner 110A includes one or more image capture devices 119A positioned about ring 117 that is designed to move along rail 118. In one embodiment, movement of movement of ring 117 can be effectuated by a stepper motor (not shown). Scanner 110A, in one embodiment, can also be provided with one or more cameras 119B.

FIG. 2A and 2B illustrate an example of a patient orientation during the scan procedure. FIG. 2A illustrates an injured body part, such as a limb, being placed on supports 114A and 114B in preparation for scanning by 3D scanner 110A. Once on support 114A and 114B, control screen 116 can be accessed to initiate 3D scanner 110A for scanning. FIG. 2B illustrates the injured body part being scanned about the limb circumferentially by the 3D scanner 110A.

For purposes of the present invention, the 3D scanner 110A can be configured to generate a substantially true image of a limb. 3D scanner 110A can be configured to interface with a computer to obtain, store, and process scanned image data. Scanned image data, in one embodiment, maybe processed using software that is capable of analyzing the data and identifying injured areas. It is contemplated that 3D scanner 110A can be scaled to allow for applications that require small dimensions, for example, mobile applications.

In some embodiments, the 3D scanner 110A can be configured to obtain color, infrared and depth information from the images collected from a scan. To that end, in one embodiment, the 3D scanner 110A can be configured with two cameras 119B. For example, a suitable camera for use with 3D scanner 110A can be the Intel RealSense SR300 camera. It is contemplated that additional configurations of cameras, infrared projectors or other imaging devices can be utilized to obtain scan data. Depending on the application, these devices can be fixedly positioned about ring 117 or can be designed to circumferentially move along ring 117. In this way, the 3D scanner 110A is capable of generating a substantially true image of a limb, and subsequently model a custom made splint based on the image data of the limb.

The 3D scanner 110A, in an embodiment, can be calibrated according to protocols using motion detection, or standards of known length to detect and correct discrepancies in data acquisition and printing. These calibration methods are well known in the art.

To facilitate the fabrication of a splint that can be customized to each individual patient, in accordance with an embodiment of the present invention, markers 120 can be placed on the limb around the injured area to help scanner 110A of the fabrication system 105 in detecting features or target areas located on the limb to which treatment, such as that provided by a therapeutic device (i.e., electrotherapeutic device) needs to focus. The markers 120, according to embodiments of the present invention, can be of different shapes, colors, and/or patterns. The markers 120, according to embodiments of the present invention, can be used to identify a site of an injury, or demarcate the desired borders for the to-be-fabricated splint. Markers 120 can also be used to provide perforation patterns or openings in the splint to allow circulation of air to the injured area to facilitate healing. Markers 120 can also be used to identify areas within the splint where thickness needs to be increased or decreased, or where the shape of the splint needs to be altered to increase or decrease pressure applied to the injured area. Markers 120 can further be used to identify where on the splint a therapeutic device, such as an electrotherapeutic device (discussed below), can be placed for treatment. Furthermore, the use of markers 120 can help, for example, to create a structural offset, spacing, or gaps between the limb and splint. The structural offset can enable the splint to, for example, reduce pressure to a target area of the limb or, accommodate a foam insert to reduce irritation, chafing, or discomfort.

The markers 120, in one embodiment, can be applied to the limb before scanning by scanner 110A. With the markers 120 in position on the limb, control screen 116 can be accessed to initiate a 3D scan by scanner 110A. As the limb is scanned, the position of the markers 120 can be captured along with the limb data as digital 3D scan data. As the digital 3D scan occurs, scanner 110A also receives information corresponding to the color spectrum, infrared profile, and depth profile of the scan, which can then be included in the 3D digital scan data file. The digital 3D scan data, including data from the markers 120, can then be transferred to a computing device and processed to construct a 3D model of a to-be-fabricated splint that conforms to the features on the limb or areas of interest identified by marker 120 where the to-be-fabricated splint needs to target.

It should be appreciated that the 3D scanned data can be processed through software of the present invention to represent a 3D model of a to-be-fabricated splint on a three dimensional coordinate system. Such a rendering of a 3D representation using the process of the present invention, can allow the user to select and manipulate the properties of specific regions on the splint prior to fabrication. The processed 3D scan data can, in one embodiment, be used in connection with various fabrication methods, for example, traditional 3D printing processes, or in connection with any fabricating devices coupled to computer interfaces.

Once the limb has been scanned, looking now at FIG. 3, in one embodiment of the present invention, a photo-polymeric material 32, and may be place over the injured body part. The photo-polymeric material 32, in an embodiment, can be a pliable, photo-curable polymeric material, such as PLA polymer, or any similar FDA approved materials. In one embodiment, the photo-polymeric material 32 can be a translucent material, such that the light can penetrate and cure the photo-curable polymeric material 32.

Placement of the photo-polymeric material 32 over the injured body part can be accomplished, in one embodiment, as illustrated in FIGS. 4A-4B by placing the injured body part between two sheets 33 of photo-polymeric material 32, such that one sheet of photo-polymeric material 32 is placed above the injured limb and another sheet of photo-polymeric material 32 placed below the injured limb. Both sheets 33 of photo-polymeric material 32, in an embodiment, can be permitted to approach each other and stick together, thus enclosing the limbs surface and adopting its volume. It should be appreciated that the sheets 33 can be secured to one another by any manner known in the art. It should be noted that although two sheets 33 of photo-polymeric material 32 are referenced in an embodiment, only one sheet 32 may be needed. In such a situation, the sheet 32 may be placed over the injured body part and then wrapped around the limb.

With the photo-polymeric material 32 placed over the injured body part, pressure can then be gently applied against the injured body part to conform the photo-polymeric material 32 to the limb. To the extent desired, another scan of the injured limb maybe executed in order to verify any variation in the injured limb's position, to ensure accuracy of the 3D scanner data to be used in fabrication.

Next, looking now at FIG. 5A, an image 30 of a to-be-fabricated splint may then be projected by scanner 110A using, for example, UV light, onto the surface of the photo-polymeric material 32. In one embodiment, as shown in FIG. 5B, image 30, having a pattern corresponding to the digitized splint processed from the 3D scanner data, may be projected from above and below (i.e., circumferentially) about the region of the injured limb on to the surface of the sheets of photo-polymeric material 32 to cure the photo-polymeric material in each sheet 32. It should be appreciated that a multitude of curable light technologies including a DLP projector or light lamp may be used to cure the photo-polymeric materials.

In an embodiment, the area of the photo-polymeric material 32 onto which UV light is projected is cured, for example in about 30 seconds or more, to form a shape of splint 40. In one embodiment, the uncured portions of the sheet of photo-polymeric material 32, can thereafter be removed to provide the customized splint 40, as shown in FIG. 5C.

It should be appreciated that customized splint 40 may need to have different properties, as will be described below, to accommodate different limb shape, profile, and/or injuries suffered by different patients. To that end, in some embodiments, the photo-polymeric material 32, can be provided with different properties, for example, throughout sheet 32, along the length of sheet 32, along the thickness of sheet 32, in each layer of sheet 32 (if sheet 32 is made from multiple layers), or a combination thereof, so that once the photo-polymeric material 32 is cured, the desired property or properties can be imparted to the resulting customized splint 40.

The customized splint 40, thereafter, can be removed from the limb for cleaning. It should be appreciated that by using two sheets of photo-polymeric material 32 placed above and below the limb, once splint 40 is formed, there is provided an upper half 41 and a bottom half 42 that can be naturally separated along an area where the two photo-polymeric sheets 33 initially adjoin, as illustrated in FIG. 6. In one embodiment, the upper half 41 and the lower half 42 of splint 40 may be separated and cleaned with biocompatible solvents, such as ethanol, to eliminate uncured portions of the photo-polymeric material 32.

As an alternative to the use of one or more sheets or photo-polymeric material 32 to fabricate customized splint 40, data obtained from the 3D scan can be utilized to fabricate the customized splint 40 by a 3D printing process. In one embodiment, instead of projecting the image of splint 40 onto the limb, as noted above, a 3D printing process may be utilized to spray or deposit (i.e., print), layer by layer, the material to form splint 40 following the desired shape and pattern, such as the pattern shown in FIG. 5C, directly onto the limb. The material, in one embodiment, can be a polymeric material such as that used above, or any other biocompatible material that can be directed through a 3D printing nozzle. To facilitate the deposition of the material on a layer by layer basis, such deposition, in accordance with in embodiment of the present invention, can be accomplished by utilizing one or more 3D printing nozzles. In one embodiment, it is contemplated that as each layer is deposited (i.e., printed), curing of the deposited material can be carried out before or as the next layer is deposited. In an embodiment, cleaning of each layer for example, by use of a solvent or solvents similar to that noted above, can be carried before or as the next layer is deposited as needed.

Of course, should it be desired, splint 40 may not need to be printed directly on the limb of the patient. Rather, splint 40 may first be printed and thereafter be placed onto the limb around the injured area.

It should be appreciated that various 3D printing protocols can be utilized in connection with the fabrication of customized splint 40 of the present invention. Examples of 3D printing protocols include 3D printing via Stereolithography (SLA), Digital Light Processing (DLP), Fused deposition modeling (FDM), Selective Laser Sintering (SLS), Selective laser melting (SLM), or Electronic Beam Melting (EBM).

The fabricated customized splint 40 may thereafter be used in an immobilization system 100 (see FIG. 7) designed to improve processes for treating broken bones and muscle injuries.

### Immobilization System

Referring now to FIG. 7, immobilization system 100 in various embodiments, may include a custom manufactured splint 40 for immobilizing an injured body part, a wireless interface 60A, and one or more therapeutic devices 50 coupled to splint 40. The therapeutic device 50 can effectuate healing of a target area by communicating with the wireless interface 60A to deliver stimulation to a targeted area.

Referring now to FIG.8A and FIG. 8B, splint 40, in one embodiment, may be configured for immobilizing an injured body part to promote proper healing. Splint 40, as noted above, can be made from one or more materials that are FDA approved, such as medical grade PLA polymer. In one embodiment, the material can be waterproof to minimize deterioration of splint 40 when exposed to perspiration, water or the like. Further, the material can be opaque, transparent, or translucent to permit light to pass through promote healing to the injured area. The material, in an embodiment, can be relatively stiff but can still be imparted with elasticity to permit some movement around the injured area. It is contemplated that the elasticity ranges can also provide the splint 40 with the capability to adopt the limb's shape and fit around the limb. The customized splint 40 may have an overall weight up to approximately 150 grams or less, 200 grams or less, 300 grams or less, 350 grams or less, 375 grams or less, 400 grams or less, 450 grams or less, and 500 grams or less. It is contemplated that the customized splint 40 may be perforated, and may include one or more patterns having uniform spaces 45, non-uniform spaces or some combination thereof in order to facilitate aeration to the injured area to minimize infection, as well as growth of mold and bacteria.

FIGS. 8A and 8B illustrate, in an embodiment, splint 40 having a multi-piece construction. FIG. 8A shows splint 40 unassembled while, FIG. 8B shows splint 40 assembled around the injured area. It should be appreciated that a multi-piece construction can provide for ease of fitting the splint 40 to a patient and ease of removal from the body part without having to disrupt the structure of the splint 40. It should be appreciated that splint 40 can also be one piece in design that can be wrapped around an injured area.

The spaces 45, in some embodiments, may provide for placement of one or more therapeutic devices, such as stimulator 50, directly against skin, while proving structure for securing the stimulators in place. As shown in FIG. 9A, a honeycomb structure provides openings 45 in the splint 40 that allow for therapeutic device 50 to be placed directly against the skin, while, as shown in FIG. 9B, providing local structure around therapeutic device 50 to secure the therapeutic device 50 in place. Splint 40, in an embodiment, may be further constructed with a grid pattern (not shown), where the grid pattern can be structured and arranged to provide one or more attributes. For example, the attributes may include having sections within the grid system that could include varying diameters of the material of the splint 40, varying space dimensions that are uniform, non-uniform or some combination thereof. It is contemplated that the grid system for the splint 40 can be structured to provide different ranges of elasticity within areas of the splint 40. At least one aspect of the grid system can include improved skin aeration during the time of healing, and can minimize itching and allergies, as well as provide access for medical staff to administer healthcare related activities, among other things.

Splint 40 can also be provided with multiple regions where the shape, thickness or size is varied to apply or relieve pressure at or around the injury site to facilitate the healing process and provide comfort to the patient. For example, in some embodiments, the shape of the splint 40 can be designed to conform or avoid contours or feature of the limb. In other embodiments, the thickness of the splint 40 can be increased to apply more pressure to the limb, or the thickness of the splint 40 can be decreased to reduce the pressure to the limb.

Splint 40, in various embodiments, may be custom manufactured using 3D technology to match the shape and size of the injured body part. In one embodiment, data obtained, for example, from a 3D scan of a limb or region of the body, can be used to model and to create a custom-fitting splint 40. In particular, the 3D scanned data can be digitally processed to create a digital representation of the limb or body region. Subsequently, in one embodiment, the splint 40 may be fabricated using a process of the present invention. In particular, the 3D scanned data may be utilized to generate a map of the customized splint 40. The map of customized splint 40 can then be projected onto a photo-curable polymer, where the polymer reacts to the projection of light, to cure the polymer in the shape of splint 40. The uncured portions are then removed and used to provide the desired customized splint 40.

Still referring to FIGS. 9A and 9B, therapeutic device 50, in various embodiments, may be configured for providing therapeutic stimulation throughout the healing process. Such stimulation can be utilized to reduce fatigue, as well as stimulate bone and muscle growth to the injured area.

In some embodiments, the therapeutic device 50 can be configured to allow control of the intensity, frequency, and duration of the stimulation. By varying the output of therapeutic device 50, user defined settings can be utilized to tailor fit treatment as needed.

As illustrated in FIG. 9A, splint 40 can have two electrodes 51, to which signals can be transmitted to stimulate an injured area. It has been contemplated that depending upon the requirements of a treatment splint 40 can be configured to accommodate a multitude of electrodes 51 and therapeutic devices 50 to stimulate the injured area.

Many patients experience atrophy of immobilized muscles over a period of time. By measuring the muscle mass index, atrophy can be monitored to guide treatment plans, and to determine the level of stimulation provided, thereby minimizing or completely preventing muscle atrophy. In an embodiment, therapeutic device 50, may also be configured as a sensor, for example, to measure the muscle mass index of the injured area. Monitoring the muscle mass index of a patient can be accomplished by sending progress data from therapeutic device 50 to wireless interface 60A. The level of stimulation delivered by therapeutic device 50 can be modulated, for instance by a clinician or patient, to meet the need of the treatment plan. Of course if desired, therapeutic device 50 can be provided with other sensor capabilities, or alternative sensor devices can be used.

In various embodiments, therapeutic device 50 can be attached to splint 40 in any suitable manner. For example, in some embodiments, a thread, clip, screw fasteners, rivets, and/or snap-fits may be used to attach therapeutic device to splint 40. In other embodiments, therapeutic device 50 can be attached to splint 40 by adhesives, bonding materials, or by being magnetically fastened.

With reference again to FIG. 7, wireless interface 60A of the present invention may also include a smart application 60B to communicate with therapeutic device 50. Wireless interface 60A in one embodiment, may be a smart device 60A, which can act as a processing unit while providing monitoring and delivery of a treatment. Additional examples of smart device 60A can include a smart phone, tablet, notebook, personal computer, a cloud network 80A based service application or any electronic devices having input output functions.

Smart application 60B, in an embodiment, may be used with existing hardware and software of wireless interface 60A. Application 60B can also be designed to control the therapeutic device 50 by sending data to device 50, as well as gather and receive data from sensors configured with device 50. Further, in an embodiment, application 60B can run programs critical to the treatment or rehabilitation plans including: rehabilitation programs, exercise programs, progress measurement reporting programs, calendar related programs, gaming programs, medical advice related programs, etc. To the extent desired, the programs can be predetermined and/or interactive in nature.

### Cloud-Based Network System or Internet

Looking now at FIG. 10, according to aspects of the present invention, the smart device application 60B can be designed to be part of a cloud network 80A to permit data storage, processing, and analytics of treatment data via the internet 80B. The smart device application 60B, in one embodiment, can be in communication with other web applications or management portals, where healthcare providers can manage patient treatments and monitor progress data. It is also contemplated that the smart device application 60B can be used to manage wireless interface 60A function and information. These and other communication protocols used by the smart device application 60B and therapeutic device 50 are known in the art.

Still referring to FIG. 10, the smart device application 60B can have a cloud network 80A connection, which can be a wireless connection or a traditional hard wired connection (i.e. via a USB cable coupled to a device connected to the cloud network 80A). In an embodiment of the present invention, a connection with the cloud network 80A can be used to: (1) transfer data from the smart device application 60B to the cloud 80A; (2) transfer data from the cloud 80A to the smart device application 60B; (3) synchronize data smart device application 60B data through the cloud 80A; (4) control the smart device application 60B from the cloud 80A; (5) Backup and restore the relevant smart phone application data. FIG. 10 shows that the user can interact and control the immobilization system 100 either from the smart phone application 60B that is downloaded onto the smart phone 60A, or from the cloud network 80A.

### Smart Device Application and Smart Device

The smart device application 60B, in an embodiment, can act as a control and processing unit for the immobilized system 100. Smart device application 60B can be designed to perform one or more of the following tasks, (1) executing the configuration, start-up and operation of devices, i.e. the therapeutic device 50; (2) data storage; (3) displaying device data, i.e. the therapeutic device 50 data, operational data, etc.; and (4) communicating with the cloud network 80A and other internet based networks 80B.

In accordance with one embodiment, smart device application 60B can communicate with the therapeutic device 50, while wireless interface 60A acts as a processing unit for data. Suitable wireless communication modalities include Wi-Fi, mobile technologies such as (G, E, 3G, H, H+ and 4G), Bluetooth or other protocols. The application 60B can utilize data encryption to provide a secure communication channel.

Application 60B can also be designed to communicate with medical software packages or other similarly related smartphone applications via the internet 80B and/or a cloud network 80A. For example, in an embodiment, application 60B also allows the physician to provide personalized care for patients by providing, for example, online treatment design, monitoring and modification of the treatment process at any time, remote control and monitoring of therapeutic device 50, analysis of progress data for each patient, and the ability to conduct a remote assessment of the patient using the phone's camera.

Referring now to FIGS. 11A-11C, smart phone application 60B can provide users with monitoring and treatment programs for therapeutic device 50 such as: (1) measuring the muscle mass index of the injured body part; (2) treatment programming capabilities by healthcare professionals; and (3) electrostimulation treatment. FIG. 11A-11C illustrate screen shots of the smart phone application 60B. FIG. 11A illustrates a screen shot 72A of the smart phone application 60B showing examples of user navigation features 71A-71E. Central home screen 71A provides user interface navigation including: patient 71B, electros 71C, treatments 71D, and session options 71E. Referring to FIG. 11B, patient navigation option 71B leads to an interface 71F where users can: access patients data, add new patients to the clinic for health care providers, see all the patient treatment data, access media uploaded by the doctor, access a patient progress window to monitor treatment plans, access a treatment window to the alter or add patient treatment plan, and access see a patient resume containing user health care records. In FIG. 11C, the electros navigation 71C option can be utilized to access the status of the therapeutic device hardware. Treatment navigation option 71D can be utilized to access the user interface to add a new treatment protocol or access and/or modify treatment protocols. Session navigation 71E option can be utilized to modify treatment sessions, including removing, adding or updating the scheduled time, duration, and desired protocol of treatment sessions.

### Stimulation Parameters and Treatment Programs

According to aspects of the present invention, the therapeutic device 50, in one embodiment, can receive signals from wireless interface 60A to deliver treatment programs to the injured area. In an embodiment, therapeutic device 50 can deliver stimulation from about 0 mA to a maximum 99 mA, with a normal operation range of 50-60 mA. Therapeutic device 50, in an embodiment, can deliver modulated waves in middle frequencies between about 1,000 Hz and about 500,000 Hz. The therapeutic device 50 may also be capable of mixing two currents (e.g., about 2000, 2500, or 4000 Hz). The therapeutic device 50 can transmit a wave form such that the current passes across 0 mA value once each period of the wave.

Exemplary user defined settings can include, low frequency stimulation, high frequency stimulation for isolated and/ or tetanic contraction. The frequency of stimulation delivered by therapeutic device 50 can include low frequency stimulation (e.g., less than 10 Hz) for isolated contractions. For example, a low frequency program can include periods of low frequency stimulation at about 2 Hz to about 10 Hz, each for about 30 seconds to about 10 minutes. The frequency of stimulation delivered by therapeutic device 50 can include high frequency stimulation (e.g., from about 25 Hz to about 100 Hz) for tetanic contractions. For example, a high frequency program can include periods of high frequency stimulation at 30 Hz and 50 Hz, each for about 30 seconds to 10 minutes. The frequency of stimulation delivered by therapeutic device 50 can include frequency stimulation from about 10 Hz to about 25 Hz for isolated and tetanic contractions. For example, a frequency program can include periods of low and high frequency stimulation from about 10 Hz and 25 Hz, each for about 30 seconds to 10 minutes. Therapeutic device 50 can deliver symmetric biphasic squared waves, for example, using frequencies of about 20 Hz to about 30 Hz, or about 30 Hz to about 50 Hz, or about 80 Hz to about 90 Hz.

Exemplary user defined stimulation treatment protocols can range from about 10 to about 15 minutes and can include periods of about 2 Hz to about 10 Hz (isolated contractions), about 10 Hz to about 25 Hz (isolated and tetanic contractions), and/or about 25 Hz to about 50 Hz (tetanic contractions). The treatment protocols can be activated by patients or by health care professionals. Electrotherapeutic device 50 can measure a user's muscle mass index before, during or after a treatment protocol. Stimulation therapy can be applied to two antagonistic muscles concurrently with one stimulation.

Another exemplary user defined treatment protocol can include two 3-10 minute sessions in the morning and evening. The average treatment time of a session can be about 5 minutes, consisting of an average treatment frequency of about 15 Hz and an average current of about 50 mA. The average time of agonistic cycle of about 200 milliseconds, followed by an antagonistic cycle of about 200 milliseconds, with an average rest cycle of 200 milliseconds between cycles. Therapeutic device 50 can measure a user's muscle mass during the rest cycle. Thermal contrast baths can be integrated into stimulation protocols to augment patient treatment. For example, the use of hot and cold baths to immerse the treated region can be utilized twice in the morning and twice in the evening, for about 1 minute in alternating hot and cold baths for up to about 6 minutes with a rest period of 30 minutes or more between thermal contrast baths.

According to aspects of the present invention, the immobilization system 100 can be operated as illustrated in FIG. 10. After the patient's injured limb has been fitted with the customized splint 40 and therapeutic device 50, the immobilization system 100 can become operational. The smart device application 60B, via wireless interface 60A, can implement treatment plans via the internet 80B and/or cloud 80A set by healthcare providers, a hospital administrator, insurance providers and others. The patient with the injured limb can interface with the smart device application 60B on the wireless interface 60A to receive and/or send via the internet 80B and/or cloud 80A, data to healthcare providers, i.e. doctors, a hospital administrator, insurance providers, and others. The smart device application 60B can be activated to communicate with the therapeutic device 50 to treat the injured area while also allowing medical staff to monitor the healing process or program from a remote location. In particular, sensor data from the therapeutic device 50 can be transmitted to the wireless interface 60A and send the sensor data subsequently sent to the healthcare provider for monitoring or additional treatment programs. It should be appreciated that although interface 60A is described as being able to wirelessly communicate with therapeutic device 50, it is contemplated that the two can communicate by a hard wire connection.

Treatment applications can be directed at health care providers with specific regulatory requirements. The immobilization system 100 can be compatible with the Health Level-7 or HL7 standard. HL7 refers to a set of international standards for transfer of clinical and administrative data between software applications used by various healthcare providers.

### Advantages

System 100 of the present invention confers many advantages over typical orthopedic treatments. For example, typical orthopedic treatments are designed to treat broken bones and muscle injuries in two linear steps. First, there is an immobilization step using a cast or a splint. Such step takes an average of 5 weeks. The second step after splint removal is a rehabilitation phase with physical therapy and suitable exercises that in average last 5 additional weeks. The total average process is 10 weeks long (FIG. 12A).

FIG. 12A and FIG. 12B illustrate a comparative timeline of healing process effects of the system 100 of the present invention (FIG. 12B) compared to the traditional healing process (FIG. 12A) timeline using plaster casts or other conventional type casts. For example, FIG. 12B shows that by combining all mentioned components in present immobilization system 100, includes the immobilization and rehabilitation phases that can be incorporated in parallel while medical staff can monitor the rehabilitation process from a distance without having unnecessary patient visits to the clinic or rehabilitation center. Instead of an average of 10 week healing process via traditional use of plaster casts or the like of FIG. 12A, with the system 100 of the present invention, healing can take about 7 weeks as shown in FIG. 12B, which allows for a faster recovery and improved quality of life for patients with injured limbs or body parts.

At least some aspects of the splint 40 can be digitally customized to fit the dimensions and imperfections of the patient's limb. It can be quickly positioned, lightweight and durable compared to the traditional casts, i.e. plaster casts and the like. Further, the splint 40 is perforated to promote air circulation, and to minimize microbial growth around the injured area. The customized splint 40 prevents muscle problems associated with traditional systems immobilization, such as the heavy weight of traditional materials. Further, the immobilization system 100 can provide stimulation can reduce pain and enhances the bone fusion process. Therefore, the customized splint saves ongoing visits to the rehabilitation center, and laborious processes for testing or consultation that instead are being performed remotely with continuous doctor supervision.

The present disclosure is directed to an immobilization system designed to improve processes for treating an injured area. Immobilization system 100 can improve patient quality of life by improving the patient's healing processes, as to eliminate unnecessary visits to hospitals or clinics, as well as saving time and money to all parties involved in the course of rehabilitation. Embodiments of immobilization system 100 of the present disclosure can be used in different industries and technologies including, the health industry, medical device technologies, space technologies, aquatic technologies, robotic system technologies and the like. Immobilization system 100 of the present disclosure can be used in creating stencils or insoles, armature or custom body protections. It is possible for the new immobilization system 100 of the present disclosure can be used in custom technology applications for to devices, such as for creating rapid protective cases, i.e. iPhone case, or a car cover replacement, a helmet or a glove.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It will be appreciated that several of the above-disclosed and other features and functions, or alternatives thereof, may be desirably combined into many other different systems or applications. All such modifications and variations are intended to be included herein within the scope of this disclosure, as fall within the scope of the appended claims.

1. A system for immobilizing and treatment of an injured area, comprising:
(a) a splint for placement around an injured area;
(b) a therapeutic device coupled to the splint to effectuate treatment to target tissues at the injured area,
(c) a wireless interface configured to communicate with the therapeutic device to transmit treatment data to the therapeutic device.

2. The system of claim 1, wherein the splint is sufficiently flexible to permit movement around the injured area.

3. The system of claim 1, wherein the splint is perforated to promote air circulation, and to minimize microbial growth around the injured area.

4. The system of claim 1, wherein the therapeutic device is designed to transmit progress data to the wireless interface.

5. The system of claim 1, wherein the therapeutic device is an electrotherapeutic device.

6. The system of claim 1, wherein the therapeutic device is also sensor to detect muscle mass index.

7. The system of claim 1, wherein the wireless interface is designed to receive treatment data from a remote location prior to the wireless interface sending the treatment data to the therapeutic device.

8. The system of claim 1, wherein the wireless interface is designed to receive sensor data from the therapeutic device for sending to a remote location for monitoring.

9. A method for orthopedic treatment, the method comprising:
(a) providing an immobilization system including:
(i) a splint, designed from a 3D scan of a limb, for placement around an injured area, and
(ii) a therapeutic device coupled to the splint and configured to effectuate treatment to target tissues around the injured area; and
(b) sending to the therapeutic device, via a wireless interface, data for treatment of target tissue around the injured area; and
(c) upon receipt of the treatment data, activating the therapeutic device to transmit a stimulation signal to the target tissues.

10. The method of claim 10, wherein the step of providing, the therapeutic device is configured to transmit data to the wireless interface.

11. The method of claim 10, wherein in the step of activating, the stimulator signal is used to treat one of skeletal tissue or muscle tissue.

12. The method of claim 10, further comprising transmitting data received by the wireless interface from the therapeutic device to a remote location to inform a health care provider of treatment progress.

13. The method of claim 10, further comprising receiving by the wireless interface, data from a remote location for additional treatment to be transmitted to the therapeutic device.

14. A method for treatment of an injured area, the method comprising:
identifying an injured region on a limb around which a splint is to be positioned;
placing markers on the limb around the injured region;
scanning the injured region having the markers to generate data for the splint to be produced that conforms to features on the limb;
fabricating the splint that conforms to the features on the limb;
attaching a therapeutic device to the splint;
wirelessly activating the therapeutic device to initiate treatment to the injured area.
